(19) United States
(12) Patent Application Publication
(10) Pub. No.: US 2018/0098769 A1
(43) Pub. Date: Apr. 12, 2018

(54) METHOD AND DEVICE FOR SELECTIVELY PROVIDING IRRIGATION FLUID TO A TARGET PATIENT TISSUE SITE

(71) Applicant: The Cleveland Clinic Foundation, Cleveland, OH (US)

(72) Inventor: Woosup Michael Park, Shaker Heights, OH (US)

(21) Appl. No.: 15/727,752

(22) Filed: Oct. 9, 2017

Related U.S. Application Data
(60) Provisional application No. 62/406,627, filed on Oct. 11, 2016.

(51) Int. Cl. A61B 17/12; A61M 3/02

(52) U.S. Cl. CPC A61B 17/12; A61M 3/0283; A61B 2217/007; A61B 2217/005; A61B 2017/12004

(57) ABSTRACT

A continuous-flow hemostatic device is provided for selectively providing irrigation fluid to a target patient tissue site. The continuous-flow hemostatic device includes a compression ring and a handle. The compression ring has a compression ring suction channel and a compression ring irrigation channel defined thereby. The compression ring has at least one suction port and at least one irrigation port on a compression ring inner wall. The suction port places the compression ring inner wall in fluid connection with the compression ring suction channel. The irrigation port places the compression ring inner wall in fluid connection with the compression ring irrigation channel. The handle has a handle suction channel and a handle irrigation channel therein. Wherein the handle suction channel is in fluid connection with the compression ring suction channel, and the handle irrigation channel is in fluid connection with the compression ring irrigation channel.

### RELATED APPLICATION

This application claims priority from U.S. Provisional Application No. 62/406,627, filed 11 Oct. 2016, the subject matter of which is incorporated herein by reference in its entirety.

### TECHNICAL FIELD

This disclosure relates to an apparatus and method for use of a continuous-flow hemostatic compression device and, more particularly, to a method and device for selectively providing irrigation fluid to a target patient tissue site.

### BACKGROUND

Certain medical procedures include providing fluid to patient tissue. For example, a medical professional can supply fluid to a procedure site, such as to sterilize, wash, or apply medication to the procedure site. It is important, yet can be difficult, to keep the provided fluid from pooling on the procedure site. A medical professional can activate an irrigator to provide fluid to the procedure site and then activate a vacuum to remove excess fluid from the site.

The procedure site can include a bleeding vessel that, in some situations, can be sutured. Prior to suturing, a medical professional may reduce the flow of blood to the blood-producing portion of the bleeding vessel. Further, it may be desirable for a medical professional to supply fluid to the procedure site for cleaning, sterilizing, applying medication, or the like. It is often desirable, yet difficult, to keep the blood from the ruptured vessel and any provided fluid, from pooling on the procedure site.

### SUMMARY

In an aspect, a continuous-flow hemostatic device is provided. The continuous flow hemostatic device includes a compression ring. The compression ring has a compression ring first surface and a compression ring second surface. The compression ring first surface is longitudinally spaced from the compression ring second surface. The compression ring has a compression ring inner wall and a compression ring outer wall. The compression ring inner wall being laterally spaced, and oppositely facing, from the compression ring outer wall. The compression ring inner wall and the compression ring outer wall both extend between the compression ring first surface and the compression ring second surface. The compression ring has a compression ring suction channel and a compression ring irrigation channel defined thereby. The compression ring having at least one suction port and at least one irrigation port on the compression ring inner wall. The suction port and the irrigation port are laterally spaced along the compression ring inner wall and oppositely facing with respect to the compression ring inner wall. The suction port places the compression ring inner wall in fluid connection with the compression ring suction channel. The irrigation port places the compression ring inner wall in fluid connection with the compression ring irrigation channel.

The continuous flow hemostatic device includes a handle. The handle has a handle proximal end and a handle distal end. The handle proximal end and the handle distal end are longitudinally spaced apart by a handle body. The handle distal end is attached to the compression ring first surface. The handle has a handle inner lumen that extends between the handle proximal end and the handle distal end. The handle inner lumen has a handle suction channel and a handle irrigation channel therein. The handle suction channel is capable of being attached to a vacuum source at the handle proximal end. The handle irrigation channel is capable of being attached to an irrigation fluid source at the handle proximal end. Wherein the handle suction channel is in fluid connection with the compression ring suction channel, and the handle irrigation channel is in fluid connection with the compression ring irrigation channel.

In another aspect, a method of selectively providing irrigation fluid to a target patient tissue site is provided. A continuous-flow hemostatic device is provided. The continuous-flow hemostatic device includes a compression ring. The compression ring has a compression ring first surface and a compression ring second surface. The compression ring first surface is longitudinally spaced from the compression ring second surface. The compression ring has a compression ring inner wall and a compression ring outer wall. The compression ring inner wall is laterally spaced, and oppositely facing, from the compression ring outer wall. The compression ring inner wall and the compression ring outer wall both extend between the compression ring first surface and the compression ring second surface. The compression ring has a compression ring suction channel and a compression ring irrigation channel defined thereby. The compression ring has at least one suction port and at least one irrigation port on the compression ring inner wall. The suction port and the irrigation port are laterally spaced along the compression ring inner wall and oppositely facing with respect to the compression ring inner wall. The suction port places the compression ring inner wall in fluid connection with the compression ring suction channel. The irrigation port places the compression ring inner wall in fluid connection with the compression ring irrigation channel.

The continuous flow hemostatic device includes a handle. The handle has a handle proximal end and a handle distal end. The handle proximal end and the handle distal end are longitudinally spaced apart by a handle body. The handle distal end is attached to the compression ring first surface. The handle has a handle inner lumen that extends between the handle proximal end and the handle distal end. The handle inner lumen has a handle suction channel and a handle irrigation channel therein. Wherein the handle suction channel is in fluid connection with the compression ring suction channel, and the handle irrigation channel is in fluid connection with the compression ring irrigation channel.

The compression ring is placed into a predetermined relationship with a target patient tissue site. The continuous-flow hemostatic device is manually forced downward to exert a predetermined amount of compressive force on the target patient tissue site with the compression ring. A vacuum source is placed into fluid connection with the handle suction channel. An irrigation fluid source is placed into fluid connection with the handle irrigation channel. Irrigation fluid is provided to the target patient tissue site, by directing the irrigation fluid from the irrigation fluid source, through the handle irrigation channel, through the compression ring irrigation channel, and out from the irrigation fluid port to the target patient tissue site. Irrigation fluid is removed from the target patient tissue site, by directing the irrigation fluid at the target patient tissue site into the suction port, through the compression ring suction channel and the handle suction channel, and into the vacuum source.

### BRIEF DESCRIPTION OF THE DRAWINGS

For a better understanding, reference may be made to the accompanying drawings, in which:

FIG. 1 is a front view of a continuous-flow hemostatic device according to one aspect of the present invention; and

FIG. 2 is a front view of an element of the aspect of FIG. 1;

FIG. 3 is a front view of another element of the aspect of FIG. 1;

FIG. 4 is a front view of a continuous-flow hemostatic device of the aspect of FIG. 1 in an alternate configuration;

FIG. 5 is a front view of an element of the aspect of FIG. 4;

FIGS. 6-8 illustrate an example sequence of operation of a portion of the aspect of FIG. 1; and

FIGS. 9-13 illustrate an example sequence of operation of a portion of the aspect of FIG. 1.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the term “patient” can refer to any warm-blooded organism including, but not limited to, human beings, pigs, rats, mice, dogs, goats, sheep, horses, monkeys, apes, rabbits, cattle, farm animals, livestock, etc.

As used herein, the term “user” can be used interchangeably to refer to an individual who prepares for, assists, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

FIGS. 1-2 depict a continuous-flow hemostatic device 100 including a compression ring 102 and a handle 104. The compression ring 102 has a compression ring first surface 106 and a compression ring second surface 108. The compression ring first surface 106 is longitudinally spaced from the compression ring second surface 108. The term “longitudinal” is used herein to indicate a substantially vertical direction, in the orientation of FIGS. 1-2. The compression ring 102 has a compression ring inner wall 110 and a compression ring outer wall 112. The compression ring inner wall 110 is laterally spaced, and oppositely facing, from the compression ring outer wall 112. The term “lateral” is used herein to indicate a direction substantially perpendicular to the “longitudinal” direction, and is shown as the horizontal direction, in the orientation of FIGS. 1-2. The compression ring inner wall 110 and the compression ring outer wall 112 both extend between the compression ring first surface 106 and the compression ring second surface 108.

As depicted in FIG. 2, the compression ring 102 has a compression ring suction channel 214 and a compression ring irrigation channel 216 defined thereby. As depicted in FIGS. 1-2, the compression ring 102 has at least one suction port 118 and at least one irrigation port 120 on the compression ring inner wall 110. The suction port 118 and the irrigation port 120 are laterally spaced along the compression ring inner wall 110 and oppositely facing with respect to the compression ring inner wall 110. That is, the suction and irrigation ports 118, 120 are diametrically opposed and face each other. The suction port 118 places the compression ring inner wall 110 in fluid connection with the compression ring suction channel 214. The irrigation port 120 places the compression ring inner wall 110 in fluid connection with the compression ring irrigation channel 216. The compression ring 102 can be formed from silicone, polyethylene, polypropylene, stainless steel, titanium, any other biocompatible material, or any combination thereof.

The handle 104 has a handle proximal end 122 and a handle distal end 124. The handle proximal end 122 and the handle distal end 124 are longitudinally spaced apart by a handle body 126. The handle distal end 124 is attached to the compression ring first surface 106. It is contemplated that the handle 104 and the compression ring 102 can be formed as a single piece, or as multiple pieces that are attached together prior to use. The handle 104 may be made out of a flexible material that would allow for the manipulation of the handle body 104 in order to place the compression ring 102 on desired patient tissue sites that would be difficult for a rigid handle 104 to access.

The handle 104 has a handle inner lumen 128 that extends between the handle proximal end 122 and the handle distal end 124. The handle inner lumen 128 has a handle suction channel 130 and a handle irrigation channel 132 therein. The handle suction channel 130 is capable of being attached to a vacuum source (not shown) at the handle proximal end 122. The handle irrigation channel 132 is capable of being attached to an irrigation fluid source (not shown) at the handle proximal end 122. The handle suction channel 130 is in fluid connection with the compression ring suction channel 214. The handle irrigation channel 132 is in fluid connection with the compression ring irrigation channel 216. The handle 104 can be formed from silicone, polyethylene, polypropylene, stainless steel, titanium, any other biocompatible material, or any combination thereof.

As shown in FIG. 3, the continuous-flow hemostatic device 100 includes a fluid path 334 for fluid to flow from the irrigation fluid source, through the handle irrigation channel 132, through the compression ring irrigation channel 216, and out from the irrigation fluid port 120 to a target patient tissue site T. The continuous-flow hemostatic device 100 includes a fluid suction path 336 for fluid to flow from the target patient tissue site T, through the suction port 118, through the compression ring suction channel 214 and the handle suction channel 130, and into the vacuum source.

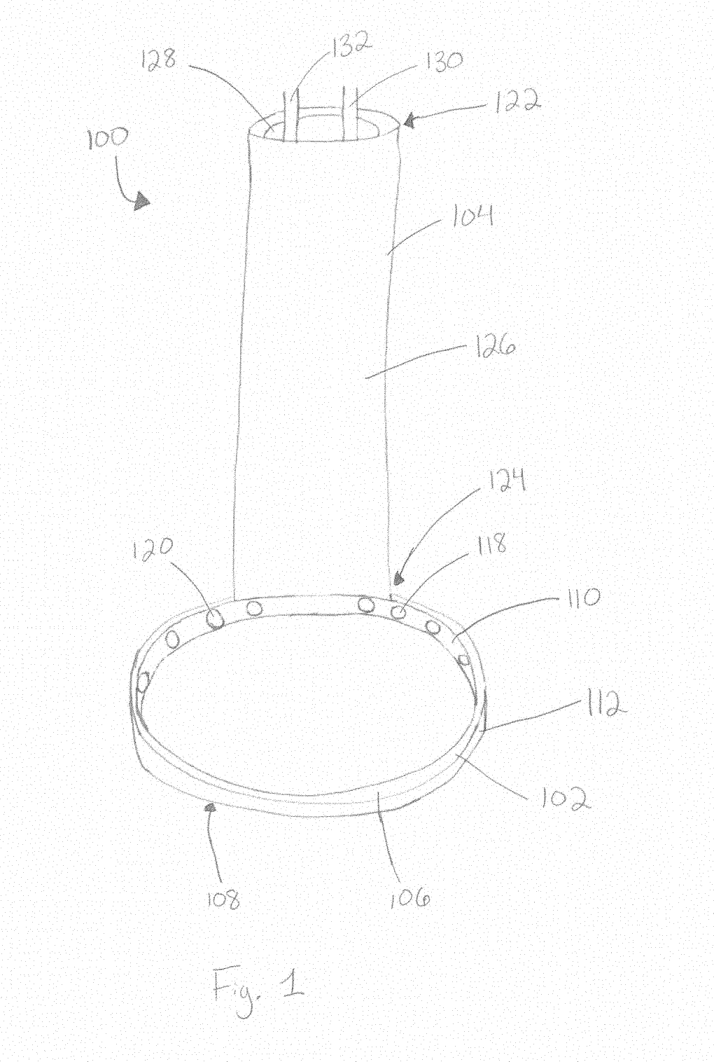

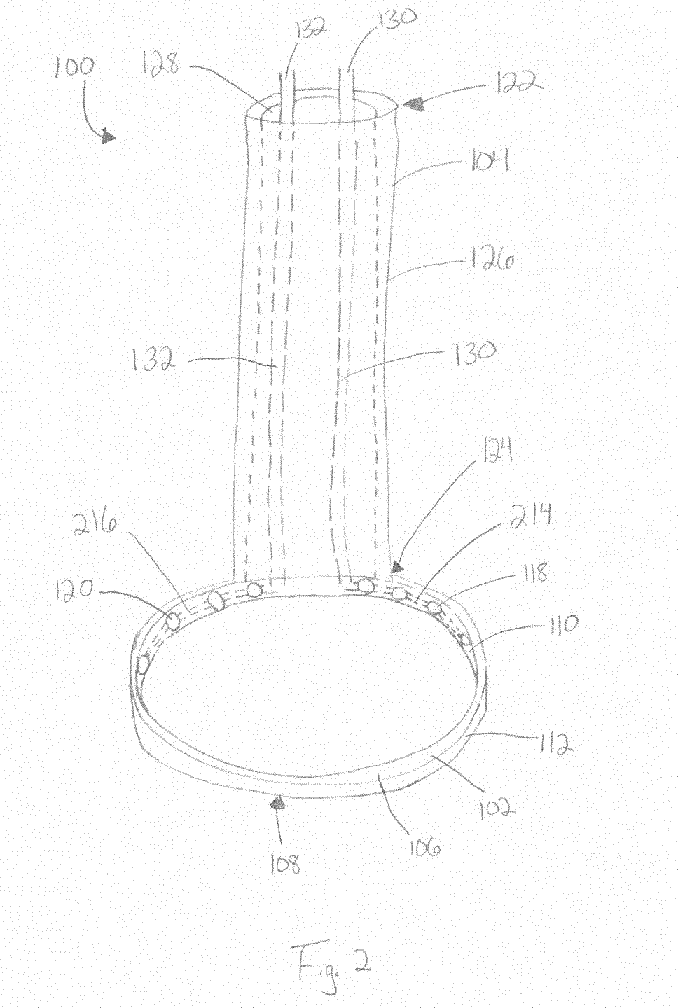

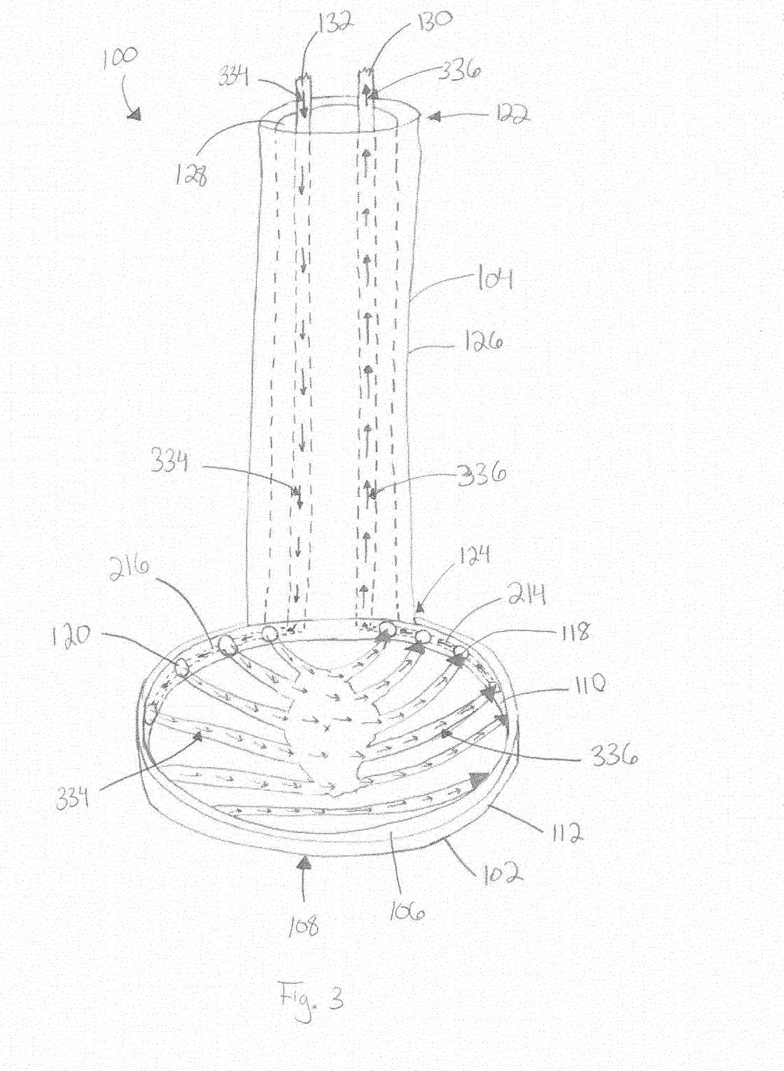

As shown in FIGS. 4-5, the continuous-flow hemostatic device 100 can include a tissue gripping ring 438. The tissue gripping ring 438 has a tissue gripping ring first surface 440 and a tissue gripping ring second surface 442. The tissue gripping ring first surface 440 has at least one securing element 444. The securing element 444 can be a single piece that laterally extends along the tissue gripping ring first surface 440, as depicted in FIG. 4. Alternatively, the securing element 444 can be positioned at several locations laterally spaced along the tissue gripping ring first surface 440, as shown in FIG. 5. The securing element 444 is capable of engaging the compression ring 102, such as by at least one of a snap-fit engagement, an adhesive engagement, any other suitable engagement, or any combination thereof. The securing element 444 can be capable of engaging the compression ring 102 without impeding the suction or irrigation ports 118, 120. That is, the securing element 444 may be configured to not be placed on or over the suction or irrigation ports 118, 120. The tissue gripping ring second surface 442 may have tissue gripping fingers 446 (the tissue gripping fingers 446 differ from a crosshatching, knurling, or other roughening/grip-enhancing surface treatment for a tissue gripping ring as known in the prior art). The tissue gripping fingers 446 are laterally spaced on the tissue gripping ring second surface 442 and extend downwardly from the tissue gripping ring second surface 442. The tissue gripping fingers 446, when present, may be provided either integrally with or separately from the tissue gripping ring 438, with one example of the latter being the provision of an insert or “cover” that is selectively associated with the tissue gripping ring second surface 442. The tissue gripping ring 438 and the tissue gripping fingers 446 can each be formed from silicone, polyethylene, polypropylene, rubber, stainless steel, titanium, any other biocompatible material, or any combination thereof.

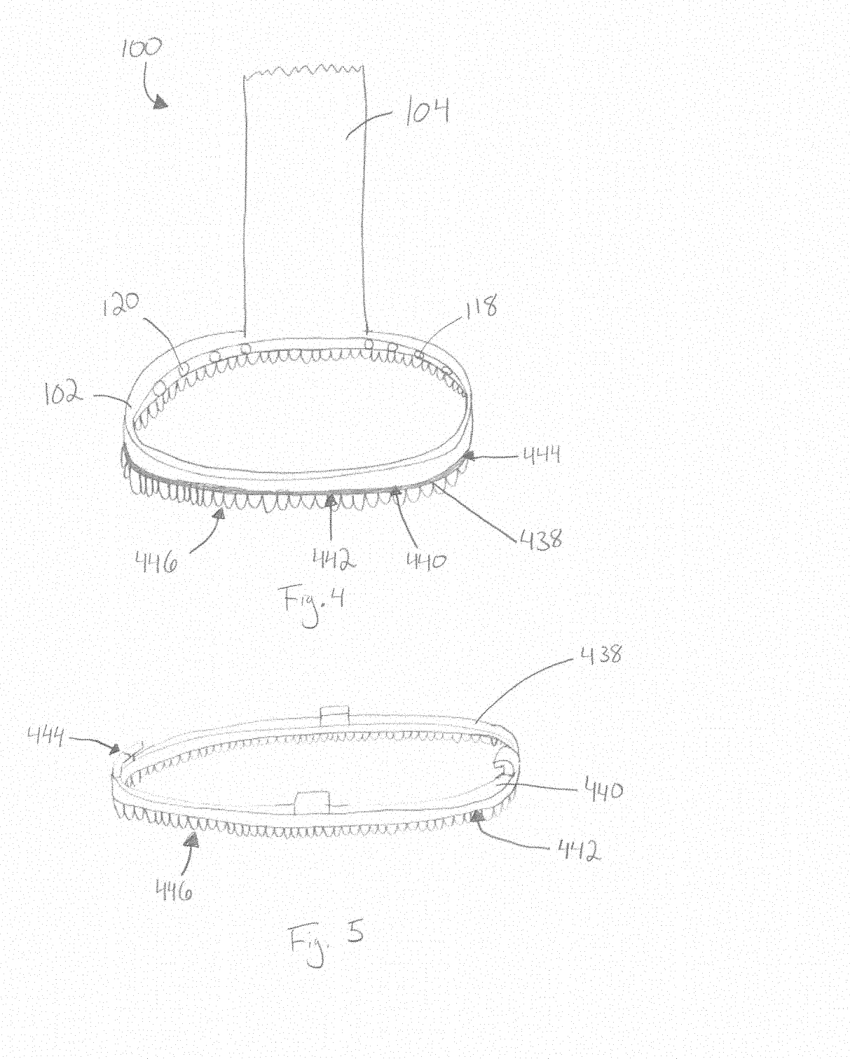

In use, the continuous-flow hemostatic device 100, as described above, may be provided to the user. As shown in FIG. 6, the user may place the compression ring 102 into a predetermined relationship with a target patient tissue site T. Once in the appropriate relationship with the target patient tissue site T, the user may manually force the continuous-flow hemostatic device 100 downward to exert a predetermined amount of compressive force on the target patient tissue site T with the compression ring 102 (shown in FIG. 7). The compressive force may be applied to reduce the flow of blood to the target patient tissue site T and/or to raise the target patient tissue site T surrounded by the compression ring inner wall 110, in relation to the patient tissue not surrounded by the compression ring inner wall 110, for suturing. However, it is to be understood that the compressive force is not limited to those applications. The term “raise” is defined herein as to lift or move (something or someone) to a higher position. If the user desires to flood the target patient tissue site T in addition to, or instead of, applying the compressive force, the user may then place a vacuum source into fluid connection with the handle suction channel 130 and an irrigation fluid source into fluid connection with the handle irrigation channel 132.

As shown in FIG. 8, the user then provides irrigation fluid to the target patient tissue site T, by selectively directing the irrigation fluid from the irrigation fluid source, through the handle irrigation channel 132 and the compression ring irrigation channel 216, and out from the irrigation fluid port 120 to the target patient tissue site T. For example, the user can use a thumb lever, a foot pedal, a push-button, or any other user-actuable device to activate the flow of fluid from the irrigation fluid source to the target patient tissue site T. The irrigation fluid directed from the irrigation fluid source floods the target patient tissue site T. The user can then remove irrigation fluid from the target patient tissue site T, by directing the irrigation fluid at the target patient tissue site T into the suction port 118, through the compression ring suction channel 214 and the handle suction channel 130, and into the vacuum source. For example, the user can use a thumb lever, a foot pedal, a push-button, or any other user-actuable device to activate the vacuum source to remove irrigation fluid from the target patient tissue site T and into the vacuum source. Although described as separate steps, the device is capable of flooding the target patient tissue T with irrigation fluid while simultaneously vacuuming the irrigation fluid away from the target patient tissue site T to create a flow-field across the target patient tissue site T.

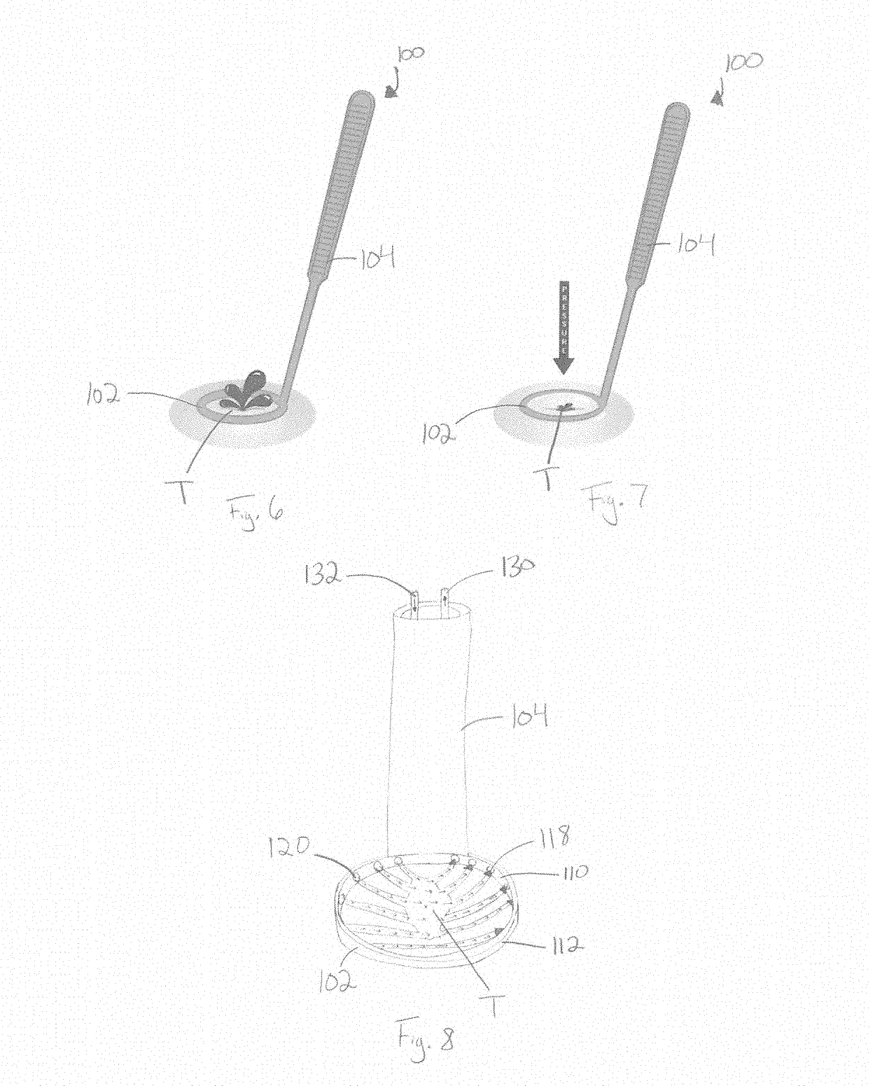

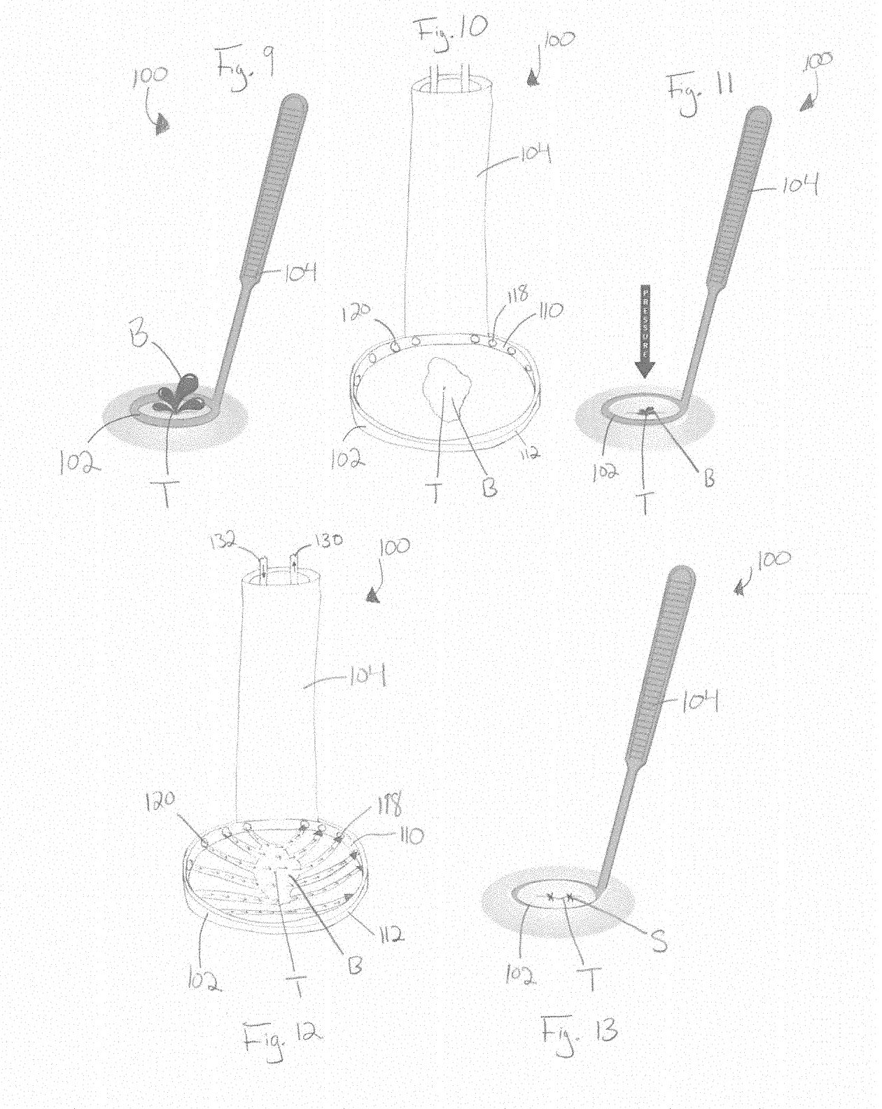

Additionally, the continuous-flow hemostatic device 100 can be used to reduce the flow and pooling of blood B at the target patient tissue site T through the use of compressive force, irrigation fluid, and/or a vacuum. This operation would be advantageous for assisting a user with reducing the flow and pooling of blood at a target patient tissue site that involves a procedure, such as suturing, on or near a blood-producing portion of a bleeding vessel. The reduction of blood at the target patient tissue site T may assist the user by increasing the visibility of the target patient tissue site T. For example, the user may place the compression ring 102 into a predetermined relationship with a target patient tissue site T, wherein a blood-producing portion of a bleeding vessel is at least partially surrounded by the compression ring inner wall 110 (shown in FIGS. 9-10). The user then manually forces the continuous-flow hemostatic device 100 downward to exert a predetermined amount of compressive force on the target patient tissue site T with the compression ring 102 to reduce the egress of blood B from the bleeding vessel (shown in FIG. 11). The compressive force will be enough to reduce the egress of blood. Further, the compressive force may cause the portion of the target patient tissue site T surrounded by the compression ring inner wall 110 to raise, in relation to the patient tissue not surrounded by the compression ring inner wall 110, which may assist the user in isolating the target patient tissue site T.

Further, the user may remove blood B, and irrigation fluid (particularly if provided by the user in the same manner as described above), from the target patient tissue site T, wherein the blood B at the target patient tissue site T is vacuumed into the suction port 118, through the compression ring suction channel 214 and the handle suction channel 130, and into the vacuum source (shown in FIG. 12). Once the blood B, and, when provided, the irrigation fluid, are removed from the target patient tissue site T, the user may then suture S the blood-producing portion of the bleeding vessel (shown in FIG. 13). Although the continuous-flow hemostatic device 100 has been described as being used in a procedure that may involve suturing, it is to be understood that the continuous-flow hemostatic device 100 may be used in any similar procedure that may involve the use of compression, irrigation, and/or a vacuum.

It is contemplated that the continuous-flow hemostatic device 100 may be used to apply a compressive force to the target patient tissue site T, in the manner as described above, wherein the operation of an irrigation fluid source or a vacuum source may not be necessary. Further, it is contemplated that the continuous-flow hemostatic device 100 may omit one or more of the suction or irrigation ports 118, 120, compression ring suction or irrigation channels 214, 216, handle suction or irrigation channels 130, 132, and/or fluid or fluid suction paths 334, 336. In this later case, however, the continuous-flow hemostatic device 100 may still be used to apply a compressive force to the target patient tissue site T, as described above.

The continuous-flow hemostatic device 100 may be configured to be used only once due to the qualities of the materials used to construct the continuous-flow hemostatic device, such as fragility and/or disposability of the material passively preventing reuse. For example, a surgeon, after a procedure, can simply discard the used continuous-flow hemostatic device 100 in any medically suitable manner (e.g., in biohazard waste as appropriate). Disposing of the continuous-flow hemostatic device 100 assists the user in preventing any unwanted consumption of time or resources that a reusable hemostatic device would consume. The disposable version of any configuration of the continuous-flow hemostatic device 100 could be made from recyclable and/or degradable plastics, such as polyethylene terephthalate, polyanhydrides, high-density polyethylene, polyvinyl chloride, low-density polyethylene, polypropylene, any other recyclable or degradable plastic, or any combination thereof.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

I claim:

1. A continuous-flow hemostatic device, comprising:
a compression ring, the compression ring having a compression ring first surface and a compression ring second surface, the compression ring first surface being longitudinally spaced from the compression ring second surface, the compression ring having a compression ring inner wall and a compression ring outer wall, the compression ring inner wall being laterally spaced, and oppositely facing, from the compression ring outer wall, the compression ring inner wall and the compression ring outer wall both extending between the compression ring first surface and the compression ring second surface,
the compression ring having a compression ring suction channel and a compression ring irrigation channel defined thereby, the compression ring having at least one suction port and at least one irrigation port on the compression ring inner wall, the suction port and the irrigation port being laterally spaced along the compression ring inner wall and oppositely facing with respect to the compression ring inner wall, the suction port placing the compression ring inner wall in fluid connection with the compression ring suction channel, the irrigation port placing the compression ring inner wall in fluid connection with the compression ring irrigation channel; and
a handle, the handle having a handle proximal end and a handle distal end, the handle proximal end and the handle distal end being longitudinally spaced apart by a handle body, the handle distal end being attached to the compression ring first surface,
the handle having a handle inner lumen extending between the handle proximal end and the handle distal end, the handle inner lumen having a handle suction channel and a handle irrigation channel therein, the handle suction channel being capable of being attached to a vacuum source at the handle proximal end, and the handle irrigation channel being capable of being attached to an irrigation fluid source at the handle proximal end;
wherein the handle suction channel is in fluid connection with the compression ring suction channel, and the handle irrigation channel is in fluid connection with the compression ring irrigation channel.

2. The continuous-flow hemostatic device of claim 1, including a fluid path for fluid to flow from the irrigation fluid source, through the handle irrigation channel, through the compression ring irrigation channel, and out from the irrigation fluid port to a target patient tissue site.

3. The continuous-flow hemostatic device of claim 2, including a fluid suction path for fluid to flow from the target patient tissue site, through the suction port, through the compression ring suction channel and the handle suction channel, and into the vacuum source.

4. The continuous-flow hemostatic device of claim 1, including a tissue gripping ring, the tissue gripping ring having a tissue gripping ring first surface and a tissue gripping ring second surface, the tissue gripping ring first surface having a securing element, the securing element capable of engaging the compression ring, the tissue gripping ring second surface having tissue gripping fingers, the tissue gripping fingers being laterally spaced on the tissue gripping ring second surface and extending downwardly from the tissue gripping ring second surface.

5. The continuous-flow hemostatic device of claim 4, wherein the securing element is capable of engaging the compression ring without impeding the suction or irrigation ports.

6. A method of selectively providing irrigation fluid to a target patient tissue site, the method comprising:
providing a continuous-flow hemostatic device including
a compression ring, the compression ring having a compression ring first surface and a compression ring second surface, the compression ring first surface being longitudinally spaced from the compression ring second surface, the compression ring having a compression ring inner wall and a compression ring outer wall, the compression ring inner wall being laterally spaced, and oppositely facing, from the compression ring outer wall, the compression ring inner wall and the compression ring outer wall both extending between the compression ring first surface and the compression ring second surface, the compression ring having a compression ring suction channel and a compression ring irrigation channel defined thereby, the compression ring having at least one suction port and at least one irrigation port on the compression ring inner wall, the suction port and the irrigation port being laterally spaced along the compression ring inner wall and oppositely facing with respect to the compression ring inner wall, the suction port placing the compression ring inner wall in fluid connection with the compression ring suction channel, the irrigation port placing the compression ring inner wall in fluid connection with the compression ring irrigation channel, and a handle, the handle having a handle proximal end and a handle distal end, the handle proximal end and the handle distal end being longitudinally spaced apart by a handle body, the handle distal end being attached to the compression ring first surface, the handle having a handle inner lumen extending between the handle proximal end and the handle distal end, the handle inner lumen having a handle suction channel and a handle irrigation channel therein, wherein the handle suction channel is in fluid connection with the compression ring suction channel, and the handle irrigation channel is in fluid connection with the compression ring irrigation channel;
placing the compression ring into a predetermined relationship with a target patient tissue site;
manually forcing the continuous-flow hemostatic device downward to exert a predetermined amount of compressive force on the target patient tissue site with the compression ring;
placing a vacuum source into fluid connection with the handle suction channel;
placing an irrigation fluid source into fluid connection with the handle irrigation channel;
providing irrigation fluid to the target patient tissue site, by directing the irrigation fluid from the irrigation fluid source, through the handle irrigation channel, through the compression ring irrigation channel, and out from the irrigation fluid port to the target patient tissue site; and
removing irrigation fluid from the target patient tissue site, by directing the irrigation fluid at the target patient tissue site into the suction port, through the compression ring suction channel and the handle suction channel, and into the vacuum source.

7. The method of claim 6, including placing the compression ring into a predetermined relationship with a target patient tissue site, wherein a blood-producing portion of a bleeding vessel is at least partially surrounded by the compression ring inner wall.

8. The method of claim 7, including manually forcing the continuous-flow hemostatic device downward to exert a predetermined amount of compressive force on the target patient tissue site with the compression ring to reduce egress of blood from the bleeding vessel.

9. The method of claim 8, including removing blood from the target patient tissue site, wherein the blood at the target site is vacuumed into the suction port, through the compression ring suction channel and the handle suction channel, and into the vacuum source.

10. The method of claim 9, including reducing the flow and pooling of blood at the target patient tissue site through the use of the compressive force, irrigation fluid, and vacuum.
